(19) United States
(12) Patent Application Publication
(10) Pub. No.: US 2013/0224777 A1
(43) Pub. Date: Aug. 29, 2013

(54) Screening method for finding samples having antiphospholipid antibodies

(71) Applicant: Juergen Patzke, Marburg (DE)

(72) Inventor: Juergen Patzke, Marburg (DE)

(73) Assignee: Siemens Healthcare Diagnostics Products GmbH, Marburg (DE)

(21) Appl. No.: 13/776,758

(22) Filed: Feb. 26, 2013

(30) Foreign Application Priority Data
Feb. 28, 2012 (EP) 12157223

(51) Int. Cl. G01N 33/86

(52) U.S. Cl. CPC G01N 33/86
USPC 435/13; 436/69

(57) ABSTRACT

The present invention is in the field of coagulation diagnostics and relates to a screening process for finding samples containing antiphospholipid antibodies (lupus anticoagulants), wherein the platelet aggregation-stimulating action of said antiphospholipid antibodies is determined in vitro.

This application claims the benefit of European Patent Application No. 12157223 filed on Feb. 28, 2012 which is incorporated by reference herewith in its entirety.

The present invention is in the field of coagulation diagnostics and relates to a screening process for finding samples containing antiphospholipid antibodies.

Antiphospholipid syndrome (APS) is one of the most common autoimmune disorders, causing thromboses, recurring miscarriages, and complications of pregnancy. Antiphospholipid syndrome is caused by “antiphospholipid antibodies” (APAs) which bind to anionic phospholipids, to proteins or to protein/phospholipid complexes. The antiphospholipid antibodies forming complexes with particular proteins and phospholipids are a very heterogeneous group of autoantibodies which may be directed against a multiplicity of antigens, for example against the apolipoprotein β2-glycoprotein I (β2GPI), cardiolipin, prothrombin, protein C, protein S, annexin V, thrombomodulin, factor XII and others, and against complexes of these proteins with phospholipids.

“Lupus anticoagulants” are antiphospholipid antibodies which by definition prolong the coagulation times of particular coagulation assays, for example of aPTT. Paradoxically, lupus anticoagulants inhibit the coagulation reaction in vitro, while an increase in the coagulation reaction (hypercoagulability) accompanies antiphospholipid syndrome (APS) in vivo. There are, however, also antiphospholipid antibodies which are not covered by these assays and which nevertheless have a prothrombotic action.

The manner in which antiphospholipid antibodies exhibit their prothrombotic action in vivo has not been elucidated conclusively. One mechanism of action seems to involve platelet activation (Urbanus, R. T. et al., Platelets and the antiphospholipid syndrome. Lupus 2008 October;17(10): 888-94).

Diagnosis of antiphospholipid syndrome (APS) in the laboratory is hampered by the heterogeneity of the antiphospholipid antibodies. Immunological methods are applied for direct detection of the antibodies. However, this also catches many antibodies that have no prothrombotic action in vivo. Coagulation assays are applied for indirect detection of the antibodies. Assays based on determining DRVVT (Dilute Russell's Viper Venom Time) in particular show relatively good correlation with the prothrombotic efficacy of antiphospholipid antibodies. Lupus diagnostics is very complex because each patient's sample must be subjected to a plurality of test steps (for an overview, see: Devreese, K. and Hoylaerts, M. F., Challenges in the diagnosis of the antiphospholipid syndrome. Clin. Chem. 2010, 56(6): 930-940).

It would therefore be desirable to have available a screening assay which enables samples to be identified that have a high probability of containing antiphospholipid antibodies with prothrombotic action. This would have the advantage that only those samples which have been shown by means of a screening assay as a first indication to contain prothrombotic antiphospholipid antibodies would have to be tested using said complex and specific detection processes.

It was therefore an object of the present invention to provide a process which enables samples to be identified that have a high probability of containing prothrombotic antiphospholipid antibodies.

This object is achieved by a process having the steps of:
a) mixing a sample from a patient with a platelet-containing reagent to give an assay mix,
b) adding a platelet activator to the assay mix, and
c) measuring platelet aggregation in the assay mix.

An increase in platelet aggregation over the normal value in the assay mix indicates the presence of prothrombotic antiphospholipid antibodies in the patient's sample.

In a mixture of a patient's sample and a platelet-containing reagent, prothrombotic antiphospholipid antibodies present in said patient's sample were found to enhance aggregation of the platelets. The assay is not specific for prothrombotic antiphospholipid antibodies, since other factors stimulating platelet aggregation, such as an elevated thrombin level for example, may also result in enhanced platelet aggregation. Nevertheless, samples that do not enhance aggregation of the platelets in the assay can be eliminated and do not have to be tested in subsequent assays for specific determination of antiphospholipid antibodies.

The term “prothrombotic antiphospholipid antibodies” refers to antiphospholipid antibodies that prolong the coagulation time of a plasma sample in vitro. A prolonged coagulation time may be determined, for example, with the aid of a DRVVT-based coagulation assay. However, such “lupus anticoagulant” assays have insufficient sensitivity, and thus some prothrombotic antiphospholipid antibodies are not detected. The process of the invention is likely to have better sensitivity because it also includes measuring the prothrombotic action in vitro. In vitro, the actual platelet aggregation-promoting action rather than the anticoagulant action is measured.

The term “patient's sample” primarily comprises body fluids, in particular platelet-rich plasma, platelet-poor plasma, serum, and whole blood.

The term “platelet-containing reagent” comprises suspensions containing functional, i.e. aggregation-capable, platelets (thrombocytes) of human origin. It may be, for example, a suspension of isolated platelets in a buffer solution. The term “suspensions” also means resuspensions of freeze-dried platelets. Alternatively, it may be platelet-rich plasma or whole blood from a donor or a plasma pool from multiple donors.

The term “platelet activator” means a substance capable of inducing platelet aggregation. Examples of suitable platelet activators are ADP (adenosine 5′-diphosphate), collagen, epinephrine, arachidonic acid, ristocetin and thrombin. It is also possible to add to the assay mix combinations of platelet activators, for example a combination of collagen and ADP (Col/ADP) or a combination of collagen and epinephrine (Col/Epi).

The preferred concentration of epinephrine in the assay mix is between 1 and 20 μmol/l. The preferred concentration of ADP in the assay mix is between 0.2 and 30 μmol/l, particularly preferably between 0.2 and 10 μmol/l.

Platelet aggregation in the assay mix, which correlates with the platelet aggregation-stimulating action of the antiphospholipid antibodies present in the patient's sample, may be determined quantitatively by measuring scattered light intensity (nephelometrically) or by measuring the turbidity of the assay mix (turbidimetrically).

Preference is given to measuring the rate of platelet aggregation in the assay mix. Measuring the rate of platelet aggregation within the period from 12 to 50 seconds after addition of a platelet activator to the assay mix has proved to be particularly meaningful. Alternatively, the maximum rate of platelet aggregation may be determined.

In another embodiment of the process of the invention, platelet aggregation is measured under flow conditions and therefore in the presence of high shear forces. Such an assay principle is implemented, for example, in the Platelet Function Analyzer System (PFA-100®, PFA-200, Siemens Healthcare Diagnostics GmbH, Marburg, Germany). This measuring process is particularly suitable in cases where the patient's sample and/or the platelet-containing reagent consists of whole blood.

To simulate such flow conditions and shear forces as prevail in relatively small arterial blood vessels, a negative pressure of about −40 mbar is generated in a special measuring cell, with the citrated whole blood present in a sample reservoir flowing through a capillary of about 100-200 μm in diameter. Said capillary leads into a measuring chamber which is closed off by a separating element, for example a membrane containing a central capillary opening (aperture) which the blood. passes through owing to the negative pressure. In most cases, the membrane is provided with one or more activators inducing platelet aggregation, at least in the area around the aperture, and consequently the blood flowing past comes into contact with the aggregation-inducing substances in the area of the aperture. Due to said induced adhesion and aggregation of the platelets, a platelet clot (thrombus) forms in the area of the aperture, which closes the membrane opening and stops the blood flow. This system usually measures the time needed until the membrane opening is closed. This “closure time” (CT) correlates with the functionality of the platelets. The patent WO 97/34698, for example, describes a measuring cell for use in a process for determining platelet function on the basis of the closure time. Preferred measuring cells are equipped with a membrane coated with collagen (Col) and additionally either ADP or epinephrine (Epi-) or coated with ADP and prostaglandin E1 (PGE1). Various separating elements and production and use thereof are described, for example, in the patent EP-B1-716744 or in EP-A1-1850134.

In a preferred embodiment of the process of the invention, the measurement of platelet activity thus comprises passing the sample which has been mixed with a platelet-containing reagent through a capillary and then through an opening of a separating element, and measuring the time required for a platelet clot to form at the opening of the separating element until the opening is closed.

When prothrombotic antiphospholipid antibodies are present in the sample, adding said sample to the platelet-containing reagent which preferably consists of whole blood shortens the closure time compared to adding a normal sample which does not contain any prothrombotic antiphospholipid antibodies (FIG. 4).

Furthermore, preference is given to determining a ratio (quotient) of the test result of the patient's sample to the test result of a normal sample (norm). A normal sample may be a sample from a healthy donor or a pool of samples from a plurality of healthy donors. For this purpose, in a second assay, a normal sample not containing any phospholipid antibodies is mixed with the platelet-containing reagent to give an assay mix, the normal platelet aggregation is measured and the ratio of the test results is formed. A ratio of greater than 1.0 of the platelet aggregation measured in the assay mix of the patient's sample to the normal platelet aggregation indicates the presence of antiphospholipid antibodies in the patient's sample.

### BRIEF DESCRIPTION OF THE DRAWINGS

FIG. 1

FIG. 1 depicts the rate of epinephrine-induced platelet aggregation (mE/s) in a sample from an obviously healthy donor in a normal plasma pool sample, and in samples from 5 patients suffering from antiphospholipid syndrome as a function of the LA1/LA2 ratio. Two different platelet-containing reagents were employed: PRP reagent A (circles) and PRP reagent B (squares). Samples from patients suffering from antiphospholipid syndrome (LA1/LA2 greater than 1) exhibit enhanced platelet aggregation over the normal samples (LA1/LA2=1).

FIG. 2

FIG. 2 depicts the ratio of the rate of epinephrine-induced platelet aggregation in a sample from an obviously healthy donor and in samples from 5 patients suffering from antiphospholipid syndrome, and the rate of epinephrine-induced platelet aggregation in a normal plasma pool sample (patient/normal plasma ratio) as a function of the LA1/LA2 ratio. Two different platelet-containing reagents were employed: PRP reagent A (circles) and PRP reagent B (squares). Samples from patients suffering from antiphospholipid syndrome (LA1/LA2 greater than 1) show an increased patient/normal plasma ratio over the normal samples (LA1/LA2=1).

FIG. 3

FIG. 3 depicts the ratio of the rate of ADP-induced platelet aggregation in a sample from an obviously healthy donor and in samples from 5 patients suffering from antiphospholipid syndrome, and the rate of ADP-induced platelet aggregation in a normal plasma pool sample (patient/normal plasma ratio) as a function of the LA1/LA2 ratio. Two different platelet-containing reagents were employed: PRP reagent C (circles) and PRP reagent D (squares). Samples from patients suffering from antiphospholipid syndrome (LA1/LA2 greater than 1) show an increased patient/normal plasma ratio over the normal samples (LA1/LA2=1).

FIG. 4

FIG. 4 indicates that antiphospholipid antibodies in a sample (lupus plasma pool, finely hatched bars) shorten the closure time in the Platelet Function Analyzer system compared to a sample (normal plasma pool, coarsely hatched bars) not containing any antiphospholipid antibodies. Four different platelet-containing reagents (1-4) of fresh whole blood from different healthy donors were employed.

### EXAMPLES

### Example 1

Measuring the platelet aggregation-stimulating action of antiphospholipid antibodies in patient's plasma

The patient's plasmas used were samples from 5 patients suffering from antiphospholipid syndrome, a sample from an obviously healthy donor, and a normal plasma pool (control plasma N, Siemens Healthcare Diagnostics Products GmbH, Marburg, Germany).

The platelet-containing reagent used was human platelet-rich plasma (PRP reagent) which had been prepared from fresh blood from obviously healthy blood donors by centrifugation (180 g, 10 min). Four platelet-containing reagents (A, B, C, D) were prepared from blood from four different donors.

135 μl of the PRP reagent were mixed with 40 μl of patient's plasma and incubated at 37° C. After five minutes, 15 μl of epinephrine solution (100 μmol/1 epinephrine) or 15 μl of ADP solution (12 μmol/1 ADP) were added to activate the platelets, and absorbance at 620 nm of the assay mix was measured turbidimetrically. The change in absorbance in the period of 12-50 seconds (mE/second) after addition of the platelet activator was determined as a measure of platelet aggregation.

All samples were tested in parallel with the aid of a commercially available assay based on a DRVVT coagulation assay for detecting lupus anticoagulants (screening reagent LA 1/confirmation reagent LA 2, Siemens Healthcare Diagnostics Products GmbH). The “LA1/LA2 ratio” of healthy donors is close to 1. An LA1/LA2 ratio markedly higher than 1 very strongly points to occurrence of antiphospholipid syndrome. The severity of said occurrence correlates with the level of the LA1/LA2 ratio.

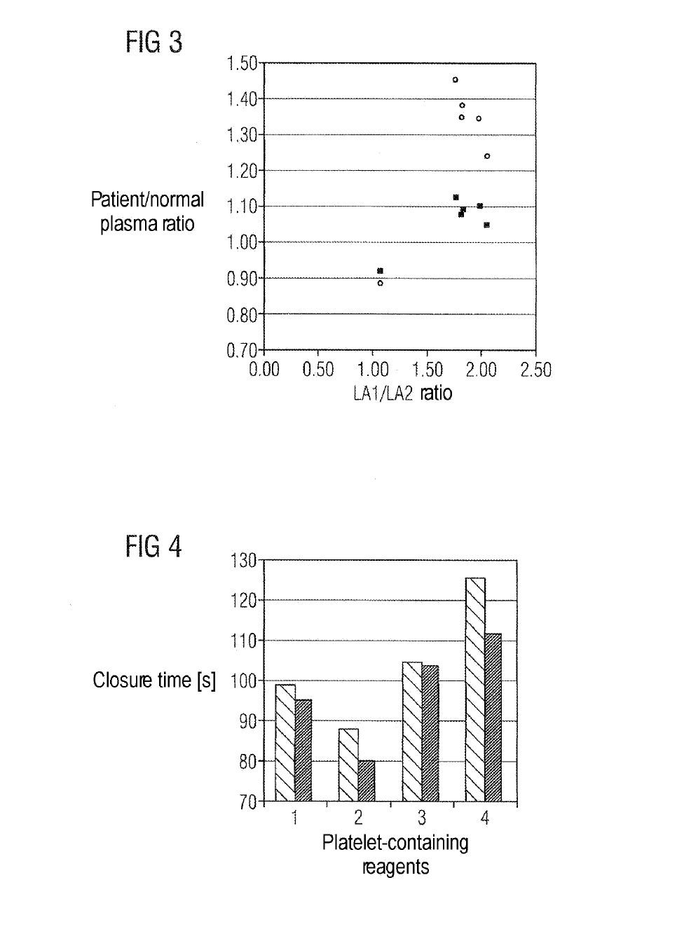

Since the rate of aggregation of platelet-rich plasma from different donors which was used here as platelet-containing reagent can vary to a relatively high degree, the results of the patient's samples were normalized by forming the quotient (“ratio”) of patient's plasma/normal plasma pool (FIGS. 2 and 3). The normalized quotient (ratio) of samples from donors with antiphospholipid syndrome is markedly higher than that for normal donors, which is close to 1 (table 1).

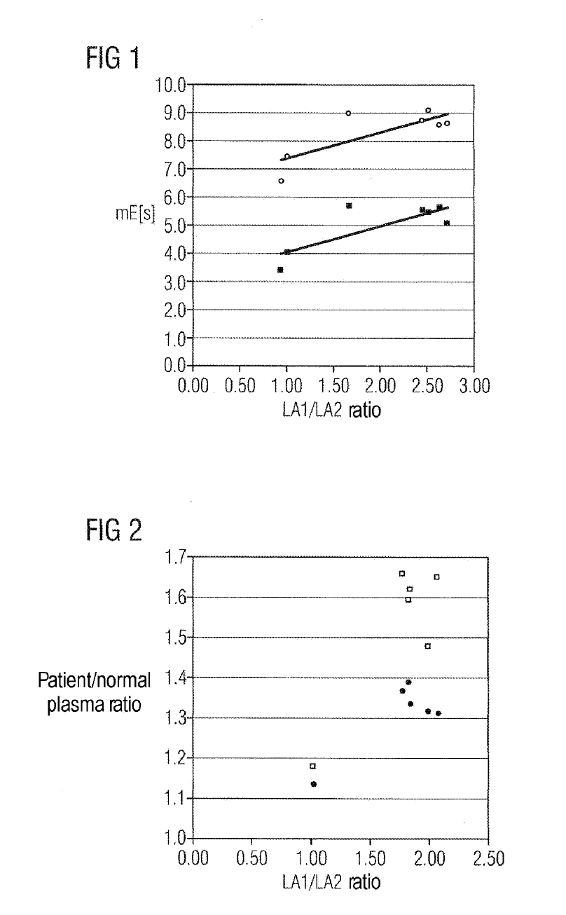

The results are depicted in FIGS. 1 to 3.

TABLE 1

| PRP | | Normal | Samples from donors with | | | |
|---|---|---|---|---|---|---|
| reagent | Activator | sample | antiphospholipid syndrome | | | |
| B | Epinephrine | 1.18 | 1.48 | 1.66 | 1.60 | 1.65 |
| C | ADP | 0.88 | 1.34 | 1.45 | 1.35 | 1.24 |

### Example 2

Measuring the closure time-shortening action of antiphospholipid antibodies in patient's plasma using the Platelet Function Analyzer

The Platelet Function Analyzer system (PFA-100®, PFA-200, Siemens Healthcare Diagnostics Products GmbH) is commonly used to aid measurement of primary hemostasis in whole-blood samples under flow conditions and therefore in the presence of high shear forces. This system measures the time needed until a membrane opening in a special measuring cell is closed (closure time) as a measure of platelet function. Prolonged closure times indicate the presence of platelet dysfunction in the sense of a reduced ability to aggregate. Shortened closure times indicate the presence of platelet dysfunction in the sense of an increased ability to aggregate.

The patient's plasma employed comprised a plasma pool of multiple plasmas from patients suffering from antiphospholipid syndrome (lupus plasma pool, with an LA1/LA2 ratio of 2.53) and a normal plasma pool (control plasma N, Siemens Healthcare Diagnostics Products GmbH).

The platelet-containing reagent used was fresh whole blood from obviously healthy blood donors. Four platelet-containing reagents (1, 2, 3, 4) were prepared from whole blood from four different donors.

The platelet-containing reagent composed of fresh whole blood was mixed with 10% (v/v) of the patient's plasma and incubated for 10 minutes. The assay mix was then pipetted into a PFA measuring cell equipped with a membrane coated with collagen (Col) and with ADP (Col/ADP), and the closure time (in s) was measured in a Platelet Function Analyzer.

The plasma pool of multiple plasmas from patients suffering from antiphospholipid syndrome (lupus plasma pool) showed shortened closure times compared to the normal plasma pool (see FIG. 4).

1. A screening process for identifying a sample which has a high probability of containing prothrombotic antiphospholipid antibodies, said method comprising the following steps:
a) mixing a sample from a patient with a platelet-containing reagent to give an assay mix,
b) adding a platelet activator to the assay mix, and
c) measuring platelet aggregation in the assay mix,
with an increase in platelet aggregation over the normal value in the assay mix indicating the presence of prothrombotic antiphospholipid antibodies in the patient's sample.

2. The process as claimed in claim 1, wherein the patient's sample consists of platelet-rich plasma, platelet-poor plasma, serum, or whole blood.

3. The process as claimed in either of the preceding claims, wherein the platelet-containing reagent consists of a suspension of isolated platelets in a buffer solution, platelet-rich plasma, or whole blood.

4. The process as claimed in any of the preceding claims, wherein a platelet activator from the group consisting of ADP, collagen, epinephrine, arachidonic acid and thrombin is added to the assay mix.

5. The process as claimed in claims 1 to 4, wherein platelet aggregation in the assay mix is measured turbidimetrically.

6. The process as claimed in claims 1 to 4, wherein platelet aggregation in the assay mix is measured nephelometrically.

7. The process as claimed in claims 1 to 4, wherein platelet aggregation in the assay mix is measured by
i) passing the assay mix through a capillary and then through an opening of a separating element, and
ii) measuring the time required for a platelet clot to form at the opening of the separating element until the opening is closed.

8. The process as claimed in claims 1 to 4, wherein the rate of platelet aggregation in the assay mix is measured, preferably within the period from 12 to 50 seconds after addition of a platelet activator to the assay mix.

9. The process as claimed in claims 1 to 4, wherein the maximum rate of platelet aggregation is measured.

10. The process as claimed in any of the preceding claims, wherein normal platelet aggregation is measured in a second assay in which a normal sample is mixed with the platelet-containing reagent to give an assay mix, and wherein the ratio of the platelet aggregation measured in the assay mix in step c) to the normal platelet aggregation is then formed, with a ratio of greater than 1.0 indicating the presence of antiphospholipid antibodies in the patient's sample.
